(19) United States
(12) Patent Application Publication
(10) Pub. No.: US 2024/0418497 A1
(43) Pub. Date: Dec. 19, 2024

(54) Relative intensity noise Cat's-eye swept source laser for OCT and spectroscopy

(71) Applicant: KineoLabs, Inc., Billerica, MA (US)

(72) Inventor: Walid A. Atia, Jamaica Plain, MA (US)

(21) Appl. No.: 18/736,769

(22) Filed: Jun. 7, 2024

Related U.S. Application Data
(60) Provisional application No. 63/521,743, filed on Jun. 19, 2023.

(51) Int. Cl. G01B 9/02055; A61B 3/10; G01B 9/02056; G01B 9/02091

(52) U.S. Cl. CPC G01B 9/02067; G01B 9/02058; G01B 9/02075; G01B 9/02091; A61B 3/102; G01B 2290/15

(57) ABSTRACT

A cat's-eye swept source laser designed for Optical Coherence Tomography (OCT) and spectroscopy, focusing on reducing relative intensity noise (RIN). It features a semiconductor gain chip and a cat's-eye configuration with an adjustable bandpass filter managed by an angle-control actuator, optimizing wavelength tuning and noise reduction. This efficient design can eliminate the need for a thermoelectric cooler, simplifying the system and reducing costs. The laser supports various imaging techniques, offering improved signal-to-noise ratios and high-resolution images.

### RELATED APPLICATIONS

This application claims the benefit under 35 USC 119(e) of U.S. Provisional Application No. 63/521,743, filed on Jun. 19, 2023, which is incorporated herein by reference in its entirety.

### BACKGROUND OF THE INVENTION

Optical coherence tomography (OCT) is a cross-sectional, non-invasive imaging modality that is used in many areas of medical imaging. For example, in ophthalmology, OCT has been widely used for imaging the retina, choroid and anterior segment. Functional imaging of the blood velocity and vessel microvasculature is also possible.

Fourier-domain OCT (FD-OCT) has recently attracted more attention because of its high sensitivity and imaging speed compared to time-domain OCT (TD-OCT), which uses an optical delay line for mechanical depth scanning with a relatively slow imaging speed. The spectral information discrimination in FD-OCT is accomplished either by using a dispersive spectrometer in the detection arm (spectral domain or SD-OCT) or rapidly scanning a swept laser source (swept-source OCT or SS-OCT).

Compared to SD-OCT, SS-OCT has several advantages, including its robustness to motion artifacts and fringe washout, lower sensitivity roll-off and higher detection efficiency.

Many different approaches have been implemented to develop high-speed swept sources for SS-OCT. One approach employs a semiconductor optical amplifier (SOA) based ring laser design (see for example Yun et al “High-speed optical frequency-domain imaging” Opt. Express 11:2953 2003 and Huber et al “Buffered Fourier domain mode locking: unidirectional swept laser sources for optical coherence tomography imaging at 370,000 lines/s,” Opt. Express 13, 3513 2005). Short cavity lasers (see for example Kuznetsov et al “Compact Ultrafast Reflective Fabry-Perot Tunable Lasers For OCT Imaging Applications,” Proc. SPIE 7554: 75541F 2010) are another example. SOA-based ring laser designs have been practically limited to positive wavelength sweeps (increasing wavelength) because of the significant power loss that occurs in negative tuning. This has been attributed to four-wave mixing (FWM) in SOAs causing a negative frequency shift in intracavity light as it propagates through the SOA (Bilenca et al “Numerical study of wavelength-swept semiconductor ring lasers: the role of refractive-index nonlinearities in semiconductor optical amplifiers and implications for biomedical imaging applications,” Opt. Lett. 31:760-762 2006).

At the same time, other architectures exist for SS-OCT that reduce the performance requirements for the swept laser source. Fechtig et al, in an article entitled Line-Field parallel swept source MHz OCT for structural and functional retinal imaging, Biomedical Optics Express 716, vol. 6, no. 3, (2015) describe a system that achieves 1 MHz equivalent A-scan rates by combining a lower sweep rate laser with a linear sensor. Even earlier examples exist such as Line-Field Optical Coherence Tomography Using Frequency-Sweeping Source by Lee et al in IEEE Journal of Selected Topics in Quantum Electronics, Vol. 14, No. 1, January 2008.

### SUMMARY OF THE INVENTION

One significant challenge in OCT is the presence of noise. One type of noise that is associated with lasers is relative intensity noise (RIN). It arises from random variations in the intensity of the laser's emission. In OCT systems employing lasers, RIN can significantly degrade the quality of the OCT images.

RIN reduction plays a role in enhancing the signal-to-noise ratio (SNR) and thus the image quality. Noise reduction techniques can broadly be classified into hardware-based and software-based techniques.

Hardware-based techniques involve improving the OCT system setup to reduce noise. Balanced detection removes the noise that is common to the original signal and the detected (like RIN), improving the SNR.

Software-based techniques involve post-processing the captured OCT images to remove noise. These can involve various digital filtering techniques, Fourier domain methods, or more advanced techniques like using wavelet transforms or total variation denoising.

Commercial ophthalmic swept source OCT systems are traditionally flying spot systems. The systems scan the eye while resolving an A-scan at every point to build up the volume image of the patients' retinas. Such systems will generally employ balanced detection and thus will typically well-suppress laser RIN.

Balanced detection cannot be realistically employed with line-field parallel swept OCT, because it would require careful balancing of two line scan cameras adding significant cost and complexity.

In general, according to one aspect, the invention features an optical coherence tomography system comprising a swept-source laser having a gain chip and tuning mechanism in a laser cavity, an interferometer receiving laser light from the swept-source laser and having a sample arm and a reference arm, a line-scan camera for detecting interference patterns formed by combining light from the reference and the sample arm, and a controller that generates and processes the interference patterns to form OCT images. A noise suppression mechanism is further provided for reducing relative intensity noise by modulating an injection current to the gain chip.

In some embodiments, a bandpass filter is provided in the laser cavity for tuning the swept-source laser. This bandpass filter can be tilt-tuned.

Preferably, the noise suppression mechanism includes a detector for detecting light from the swept-source laser for feedback to control the injection current.

The injection current can be modulated using a proportional-integral-derivative controller.

The noise suppression mechanism might further control the injection current based on a power reference curve during a wavelength sweep of the swept-source laser.

In general, according to another aspect, the invention features a method for reducing relative intensity noise (RIN) in a swept-source laser for OCT. The method comprises generating an output beam with swept laser including a gain chip in a laser cavity, sweeping a wavelength of the swept laser using a bandpass filter, monitoring a power of the output beam using a detector, and modulating an injection current to the gain chip based on the power of the output beam.

In general, according to another aspect, the invention features an optical coherence tomography system comprising a swept-source laser having a gain chip and tuning mechanism in a laser cavity in which the gain chip is operated in coolerless configuration with no thermoelectric cooler.

In general, according to another aspect, the invention features an optical coherence tomography system comprising a swept-source laser having a gain chip and tuning mechanism in a laser cavity, an interferometer receiving laser light from the swept-source laser and having a sample arm and a reference arm, a line-scan camera for detecting interference patterns formed by combining light from the reference and the sample arm, a beam splitter receiving light in free space from the swept source laser, a detector for detecting light from the beam splitter, and a ridge injection current driver for modulating an injection current to the gain chip based on the response of the detector.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIGS. 1 and 2 are schematic side views of a cat's-eye tunable laser in a line-field parallel swept OCT system; and

FIG. 3 is a plot of the laser frequency, angle of the angle control actuator 132, power set point reference signal 250, RIN or error signal for the collection of the line interference signals for a B-scan.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

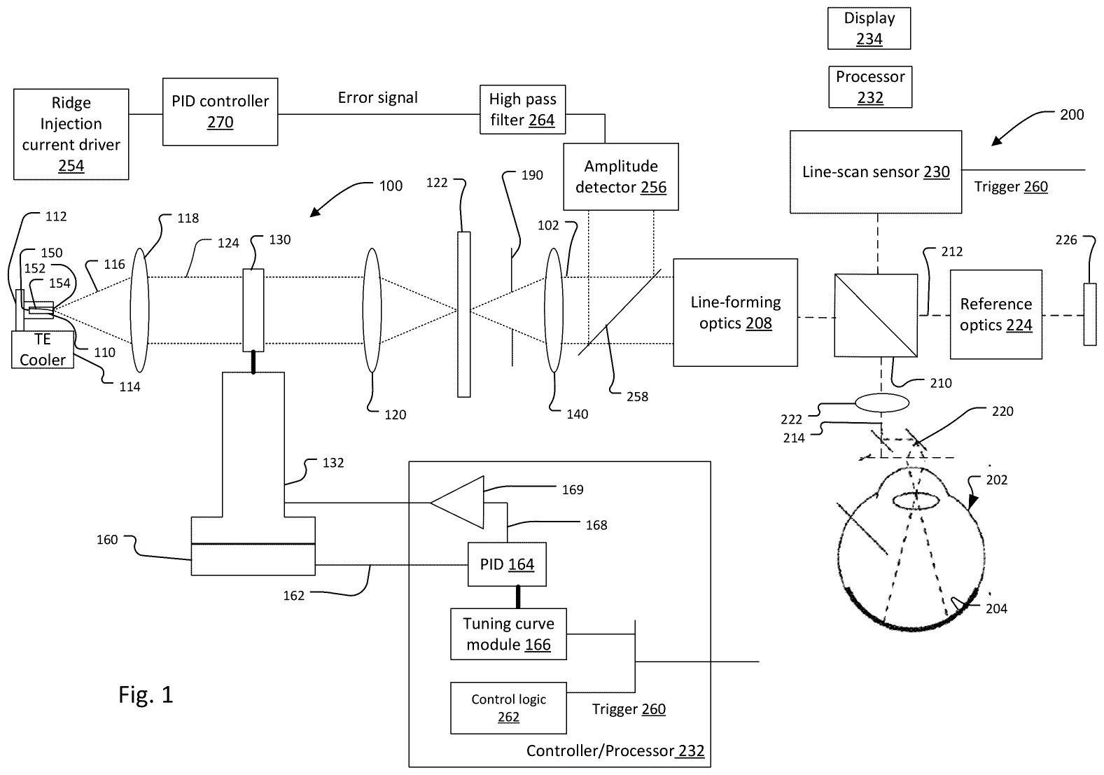

FIG. 1 shows a line-field parallel swept OCT system 200 with a tunable laser 100, which has been constructed according to the principles of the present invention.

The illustrated example shows the specific implementation of an interference filter tuned cat's eye laser. In more detail, the laser's amplification is provided by a GaAlAs gain chip 110, in one example. The gain chip 110 amplifies light in the wavelength range of about 800 to 900 nanometers. Preferably its center wavelength is around 840 nanometers, which is useful for applications such as ophthalmic imaging and other diagnostic uses because of the water window (650 to 950 nm) at these wavelengths. Another advantage of this wavelength range is that it can be detected with standard cameras with silicon-based imager chips. Specifically, the output is detected with silicon, e.g., complementary metal-oxide-semiconductor (CMOS) or charge-coupled device (CCD), imagers.

Other material systems can be selected for the gain chip, however. Common material systems are based on III-V semiconductor materials, including binary materials, such as GaN, GaAs, InP, GaSb, InAs, as well as ternary, quaternary, and pentenary alloys, such as InGaN, InAlGaN, InGaP, AlGaAs, InGaAs, GaInNAs, GaInNAsSb, AlInGaAs, InGaAsP, AlGaAsSb, AlGaInAsSb, AlAsSb, InGaSb, InAsSb, and InGaAsSb. Collectively, these material systems support operating wavelengths from about 400 nanometers (nm) to 2500 nm, including longer wavelength ranges extending into multiple micrometer wavelengths. Semiconductor quantum well, quantum cascade and quantum dot gain regions are typically used to obtain especially wide gain and spectral emission bandwidths, and support operation up to 250 μm in wavelength. Quantum well layers may be purposely strained or unstrained depending on the exact materials and the desired wavelength coverage.

When longer wavelengths lasers are used, the InGaAs imager chips would often be used.

In the preferred current embodiment, the gain chip 110 is mounted in a TO-can type hermetic package 112. This protects the chip 110 from dust and the ambient environment including moisture. In some examples, the TO-can package has an integrated or a separate thermoelectric cooler (TEC) 114. Other mounting approaches can of course be used such as mounting in a windowed butterfly package.

In the preferred embodiment, the chip 110 is operated in a coolerless configuration. No TEC cooler is used. This is possible since the laser operates very efficiently and the InGaAs chip is very power efficient.

The chip 110 is preferably a single angled facet (SAF) edge-emitting chip. As such, it has a high reflectivity (HR) coated rear facet 150. It has an antireflective (AR) coated front facet 152. In addition, for improved performance, it has a curved ridge waveguide 154 that is perpendicular to the rear facet 150 but is angled at the interface with the front facet 152. This angling at the front facet along with the AR coating reduces reflections at the front facet reflectivity by up to 40 dB and significantly improves laser performance by reducing parasitic reflections that can otherwise lead to non-smooth tuning and mode-hopping.

The free space beam 116 from the package 112 is diverging in both axes (x, y). It is collimated by a collimating lens 118. The resulting collimated beam is received by a cat's eye focusing lens 120, which focuses the light onto a cat's eye mirror/output coupler 122. This defines the other end of the laser cavity, extending between the mirror/output coupler 122 and the back/reflective facet of the gain chip 110.

The collimated light 124 between the collimating lens 118 and the cat's eye focusing lens 120 passes through a thin film interference bandpass filter 130. This provides a pass band of approximately 0.3 nanometers (nm) full width at half maximum (FWHM) for OCT applications. More generally, its pass band is between 0.2 nm and 0.5 nm FWHM, or more generally between 0.1 nm and 2 nm FWHM. Even more generally, it is between 0.05 nm to 5 nm FWHM.

The bandpass filter is held on an arm of an angle control actuator 132 that changes the angle of the bandpass filter 130 to the collimated light 124. In one example, the angle control actuator is a galvanometer. In other examples, the angle control actuator 132 is a servomotor or an electrical motor that continuously spins the bandpass filter 130 in the collimated beam 124. This allows for tilting of the bandpass filter 130 with respect to the collimated beam 124 to thereby tilt-tune the filter and thus change the passband to scan or sweep the wavelength of the swept laser 100.

Tuning speed specifications for a galvanometer generally range from 0.1 Hz to 50 KHz. For the higher speeds, a 25 kHz resonant galvanometer can be used with bi-directional tuning, but higher and lower speeds can be used. Wavelength tuning speed is usually given in nm/sec, so for a 100 Hz tuning speed ideal for retinal imaging applications where a line-speed camera at 10 0kHz will give 1000 sampled bandwidth points and 70 nm tuning range, this would give 70 nm/10 msec=7000 nm/sec. In general, the tuning speed should be between 3,000 nm/sec and 11,000 nm/sec or higher.

For retinal or industrial imaging with low-cost CMOS or CCD cameras, 840 nm center wavelength is an ideal water window. The tuning range is usually minimally 30 nm. Preferably, the tuning range is closer to 60 nm or 70 nm or more. This provides good resolution of <8 micrometers in air. In general, the tuning range should be between 30 nm and 100 nm.

The size of the collimated beam 124 is important for many applications. As a general rule, a smaller beam results in higher divergence resulting in a larger cone half angle (CHA). This reduces the minimum line width over angle for a filter. In the current embodiment, the collimated beam is preferably not less than, i.e., greater than, 1 millimeter (mm) FWHM and is preferably greater than 2 mm FWHM for retinal OCT application. It can be smaller, however, for many spectroscopy applications in the infrared, visible or ultraviolet. In general, the CHA should be less than 0.04×0.02 degrees in the two axes perpendicular to the beam's axis and preferably about 0.02×0.01 degrees or less.

The light from the gain chip is polarized. In the common architectures, the polarization is horizontal or parallel to the epitaxial layers of the edge-emitting gain chip 110. In the preferred configuration, the filter is oriented to receive the S polarization in order to maintain narrow line width of the filter as it is tilt tuned. On the other hand, the P polarization broadens drastically at large tilt angles. S polarization has higher loss at larger tilt angles than P. So, the filter design needs to address these issues by providing a low enough loss across the tuning band for S, in the current embodiment.

On the other hand, for spectroscopy, P polarization configurations might be desirable due to the higher powers across the scanband.

In general, the present cat's-eye configuration provides a number of advantages. It provides low loss, low tolerance, repeatable stable operation since it provides for a lower angle wavelength change over grating-based lasers.

The mirror/output coupler 122 will typically reflect about 80% of the light back into the laser's cavity and transmit about 20% of light. More generally, the mirror/output coupler can reflect from 10% to 99% of light (transmitting 90% to 1%, respectively), depending on the output power and laser cavity loss desired. Higher reflectivity results in lower loss cavities and thus wider laser tuning range where gain exceeds loss, but results in lower output power. In typical operation, the mirror/output coupler 122 reflects less than 90%.

In some embodiments, an iris or mask 190 is added typically after the mirror output coupler 122 to clip the beam edge. This reduces power fluctuations as the beam wanders due to refraction in the tilting bandpass filter 130. Preferably, it is between 80% and 95% and preferably about 90% of the FWHM beam size.

Typically, the diverging beam from the mirror output coupler 122 is typically collimated with an output collimating lens 140 to form a free space output beam 102.

The angle control actuator 132 is operated as a servomechanism. In the illustrated embodiment, the angle control actuator 132 is a servo-controlled galvanometer with an angle encoder 160. The angle encoder 160 produces an angle signal 162 indicating the angle of the galvanometer and thus the filter 130 to the collimated beam 124. Preferably, the encoder is an optical encoder and is often analog, but is digital in some examples.

A controller/processor 232 receives the angle signal 162 at a PID (proportional-integral-derivative) controller 164. The PID controller 164 compares the angle signal 162 to a specified tuning function stored in the tuning curve module 166. Often, the desired tuning curve is stored in a look up table or is generated algorithmically. Often this is an approximately sawtooth or triangular waveform. The PID controller 164 produces the control function 168 that is used to drive the windings of the galvanometer 132 via an amplifier 169 to minimize the difference between the angle signal 162 and the tuning curve.

In the illustrated example, the OCT system 200 is employed for ophthalmic analysis of a human eye 202 and specifically the retina 204. Although in some cases, it is use for anterior chamber analysis of the eye.

Light in the form of free space beam 102 from the laser 100 passes in free space to line-forming optics 208 and then to a beamsplitter 210, such as a cube beamsplitter, of the OCT interferometer.

Typically, the line-forming optics 208 includes one or more cylindrical lenses and possibly several additional lenses in a beam expander configuration. The line forming optics 208 converts the light from the laser 100 into a line or more specifically a rectangular profile with an aspect ratio of at least 10 to 1 and typically greater than 100:1, and often 400:1, or more. That is, when looking along its optical axis, the light from the line-forming optics 208 has a line or more specifically a rectangular two-dimensional profile that is at least 10 times longer in one dimension than the other dimension, measuring at FWHM.

The beamsplitter 210 divides the light between the reference arm 212 and the sample arm 214 in the illustrated Michelson arrangement. The light propagates in free space between one or more lenses that form projection and collection optics 222 in the sample arm and illuminates the sample 202, a typical sample being tissues, e.g. retina, 204 in the human eye.

The light is scanned across the sample, typically with a galvanometer driven scanning mirror 220 between beamsplitter 210 and the sample 202. The scanning mirror scans so that the beam of light is moved in the direction that is orthogonal to the major axis of the rectangular beam profile.

Light in the reference arm 212 is conditioned by one or more lenses of reference arm optics 224 and reflected by reference mirror 226.

The collected sample light received back through the projection and collection optics 222 is combined with reference arm light to form light interference on a line-scan camera or sensor 230. The line-scan sensor typically has a linear array of at least 512 pixels, and often at least 1024 or 2048 pixels to detect interference signals for a line.

An important aspect of the illustrated example is that the light from the cat's-eye swept laser 100 and specifically the gain chip 110 travels through the entire OCT interferometer to the line-scan sensor 230 without any optical fiber in the light path. The free space extends between the cube beamsplitter 210, and the lenses of the line-forming optics 208, collection optics 222, reference arm optics 224. No waveguides, such as optical fiber, need to be present.

The output from the sensor 230 corresponding to the detected interference pattern is readout by a processor 232. The results can be stored in the processor and/or displayed on display 234. The Fourier transform of the interference light at the different wavelengths or frequencies of the swept laser 100 reveals the profile of scattering intensities at different path lengths, and therefore scattering as a function of depth (z-direction) in the sample (see for example Leitgeb et al, “Ultrahigh resolution Fourier domain optical coherence tomography,” Optics Express 12(10):2156 2004). The profile of scattering as a function of depth for a point is called an axial scan (A-scan). The combination of the projected line and line-scan sensor 230 produces a cross-sectional image (tomogram or B-scan) of the sample. As the projected line is scanned across the eye by the scanning mirror 220, a collection of B-scans are acquired creating a data cube or cube scan.

According to the invention, a portion, typically less than 10%, of the output beam 102 is picked-off and directed by a beam splitter 258 to an amplitude detector 256. In this way, the power in the output beam 102 is monitored. Specifically, the high frequency fluctuation in the power due to RIN and other sources of amplitude noise are detected by the amplitude detector 256.

A high pass filter 264 filters the electrical signal from the amplitude detector to produce an error signal for an injection current PID controller 270. This controls a ridge injection current driver 254, which is often a voltage to current amplifier that provides the current to the ridge 154 of the chip 110. Cutoff for the high pass filter 264 is typically between 100 Hz and is often less than 5000 Hz.

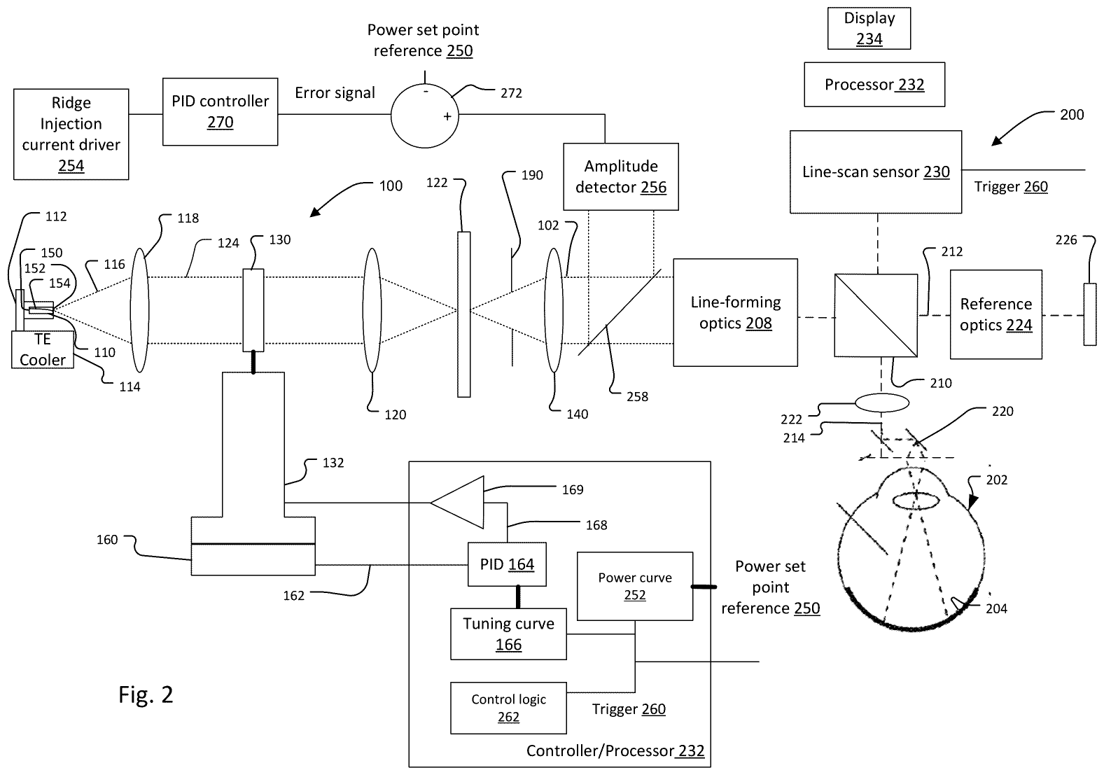

FIG. 2 shows another example of the RIN suppression feedback to the chip 110. Here, the amplitude signal from the amplitude detector 256 is received at one terminal of a comparator 272. This is compared to a power set point reference signal 250 that is produced by a power curve module 252 that can be a process executing on the controller processor 232. This power curve module 252 produces the power reference 250 signal from a look up table or algorithmically in response to the trigger 260. This comparison produces the error signal that is received by the PID controller 270 to control the ridge injection current driver 254.

The ridge injection current driver 254 modulates the current to the ridge 154 of the chip 110 to maintain a stable power in the output beam 102.

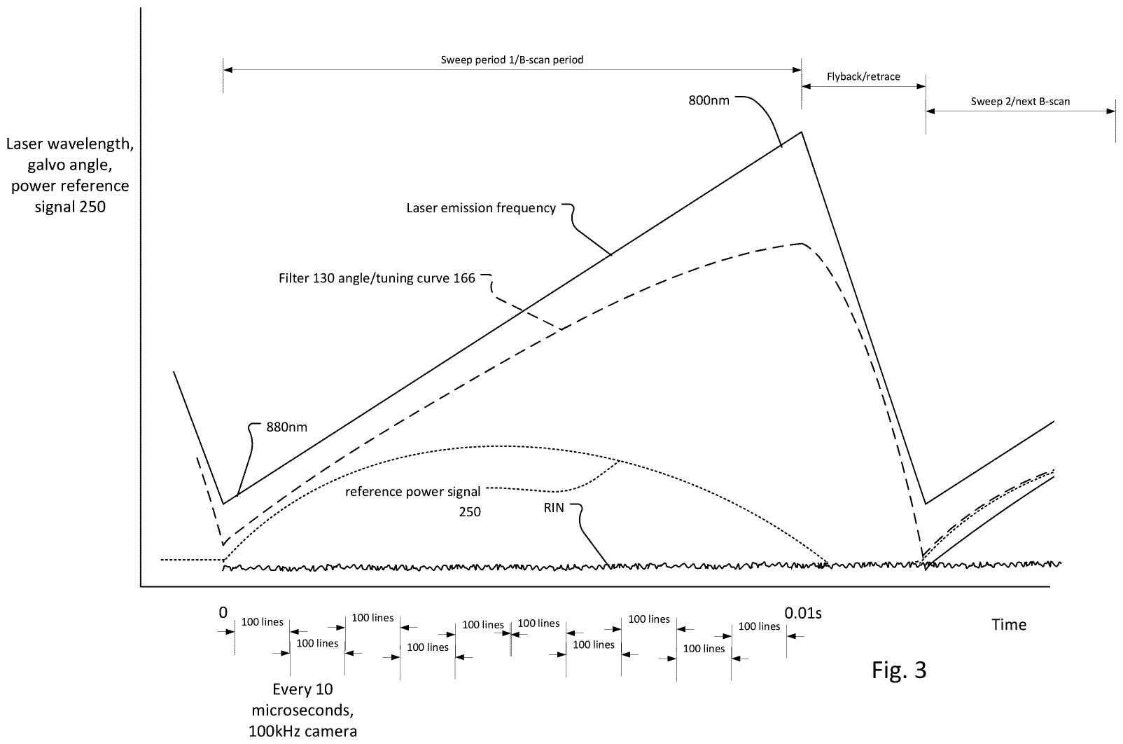

FIG. 3 is a plot of the laser frequency, angle of the angle control actuator 132, power reference set point signal 250, RIN or error signal for the collection of the line interference signals needed for a B-scan.

In more detail, the control logic 262 triggers the beginning of the scan with the trigger 260. The laser 100 is swept in wavelength so that its frequency changes preferably linearly with time over the sweep or B-scan period. A linear frequency sweep is often desirable because it allows for efficient use of the camera sample rate and avoids the need to resample.

For the linear sweep in frequency, the angle of the filter 130 must be tuned in a non-linear fashion. As shown, the rate of change of the angle of the filter 130 slows with increasing angle and shorter wavelengths, higher frequency of the laser emission 102. This tuning curve for linear frequency sweeping is produced by and/or stored in the tuning curve module 166.

As the laser 100 begins its sweep, the control logic 262 of the controller-processor 232 generates the trigger signal 260 that initiates the capturing of the line interference signals as the laser tunes. Preferably, at least 250 line interference signals are captured by the line-scan camera 230 within the sweep period of the laser 100. Currently more than 500 line interference signals, such as 1000 line interference signals or more are captured across the wavelength sweep of the laser.

Preferably, the sweep period is less than 0.05 seconds. In the current examples, a sweep period of about 0.01 seconds or less provides acceptable B-scans of the human eye despite microsaccades and other movement.

In a current implementation, the line-scan camera 230 operates as about 100 kLPS (lines per second) for a sweep period of about 0.01 seconds. This informs the requirements for the RIN cancellation. The RIN cancellation only needs to be within the Nyquist frequency of the line-scan camera line speed. For example, a 100 kLPS camera speed will have a Nyquist frequency of 50 kHz where the sampling is relevant. Therefore, the loop bandwidth of the feedback control of the ridge injection current is at least 10% of the line rate of the line-scan camera and is preferably about 50% of that line rate and can be 100% or more of the line rate.

In embodiment of FIG. 2, the ridge injection current or power to the chip 110 is also modulated across the sweep period. The reference power signal 250 provided by the power curve module 252 generates a signal that will increase the power to the chip 110 in the center of the sweep compared to the start and end of the sweep. This curve will typically approximate a Hanning, Hamming, or Gaussian window. This improves the performance by keeping the average power lower while maximizing power in the portions of the sweep that are important for the windowed FFT math performed by the processor 232 to generate the B-scans.

While this invention has been particularly shown and described with references to preferred embodiments thereof, it will be understood by those skilled in the art that various changes in form and details may be made therein without departing from the scope of the invention encompassed by the appended claims.

What is claimed is:

1. An optical coherence tomography system comprising:
a swept-source laser having a gain chip and tuning mechanism in a laser cavity;
an interferometer receiving laser light from the swept-source laser and having a sample arm and a reference arm;
a line-scan camera for detecting interference patterns formed by combining light from the reference arm and the sample arm;
a controller for generating and processing the interference patterns to form OCT images; and
a noise suppression mechanism for reducing relative intensity noise by modulating an injection current to the gain chip.

2. The system as claimed in claim 1, further comprising a bandpass filter in the laser cavity for tuning the swept-source laser.

3. The system as claimed in claim 2, wherein the bandpass filter is tilt tuned.

4. The system as claimed in claim 1, wherein the noise suppression mechanism includes a detector for detecting light from the swept-source laser for feedback to control the injection current.

5. The system as claimed in claim 1, wherein the injection current is modulated using a proportional-integral-derivative controller.

6. The system as claimed in claim 1, wherein the noise suppression mechanism further controls the injection current based on a power reference curve during a wavelength sweep of the swept-source laser.

7. A method for reducing relative intensity noise (RIN) in a swept-source laser for OCT comprising:
generating an output beam with swept laser including a gain chip in a laser cavity;
sweeping a wavelength of the swept laser using a bandpass filter;
monitoring a power of the output beam using a detector;
modulating an injection current to the gain chip based on the power of the output beam.

8. The method as claimed in claim 7, further comprising using a bandpass filter in the laser cavity for tuning the swept laser.

9. The method as claimed in claim 8, wherein the bandpass filter is tilt tuned.

10. The method as claimed in claim 7, further comprising using the monitored power as feedback to control an injection current of the gain chip.

11. The method as claimed in claim 7, wherein the injection current is modulated using a proportional-integral-derivative controller.

12. The method as claimed in claim 7, further comprising controlling the injection current based on a power reference curve during a wavelength sweep of the swept-source laser.

13. An optical coherence tomography system comprising:
a swept-source laser having a gain chip and tuning mechanism in a laser cavity;
an interferometer having a sample arm and a reference arm;
a line-scan camera for detecting interference patterns formed by combining light from the reference arm and the sample arm; and
a controller for generating and processing the interference patterns to form OCT images;
wherein the gain chip is operated coolerless with no thermoelectric cooler.

14. An optical coherence tomography system comprising:
a swept-source laser having a gain chip and tuning mechanism in a laser cavity;
an interferometer receiving laser light from the swept-source laser and having a sample arm and a reference arm;
a line-scan camera for detecting interference patterns formed by combining light from the reference arm and the sample arm;
a beam splitter receiving light in free space from the swept-source laser;
a detector for detecting light from the beam splitter;
a ridge injection current driver for modulating an injection current to the gain chip based on the response of the detector.
